(19) United States
(12) Patent Application Publication
(10) Pub. No.: US 2005/0124591 A1
(43) Pub. Date: Jun. 9, 2005

(54) Use of vitamin Ds to treat kidney disease

(76) Inventors: Jin Tian, Waukegan, IL (US); Joel Melnick, Wilmette, IL (US); Laura Williams, Gurnee, IL (US); Leticia Delgado-Herrera, Lake Forest, IL (US); Ping Qiu, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/974,243

(22) Filed: Oct. 27, 2004

Related U.S. Application Data
(63) Continuation-in-part of application No. 10/901,660, filed on Jul. 28, 2004.
(60) Provisional application No. 60/491,025, filed on Jul. 29, 2003.

(51) Int. Cl. A61K 31/59; A61K 31/401; A61K 9/70

(52) U.S. Cl. 514/167; 424/449; 514/423

(57) ABSTRACT

Disclosed are compositions containing a VDRA/Vitamin D analog to treat or prevent kidney disease, including chronic kidney disease. The present invention also relates to methods of treating kidney disease by administering to a patient a pharmaceutical composition containing a therapeutically effective amount of a VDRA/Vitamin D analog. Compositions according to the invention include a VDRA/Vitamin D analog and at least one of the following agents: an ACE inhibitor, an angiotensin (II) receptor blocker (ARB) and aldosterone blocker in therapeutically effective amounts to inhibit renin production or inhibit activation of the renin-angiotensin-aldosterone system. Preferred compositions contain paricalcitol with at least one of these other agents. Such combinations can avoid ACE inhibition escape and aldosterone escape with subsequent increase in angiotensin (II) and aldosterone generation.

### CROSS REFERENCE TO RELATED APPLICATIONS

The present application claims priority to U.S. application Ser. No. 10/901,660, filed on Jul. 28, 2004 which claims priority to U.S. Provisional Application No. 60/491,025, filed on Jul. 29, 2003, hereby incorporated in its entirety by reference.

### FIELD OF THE INVENTION

The present invention relates to the use of a Vitamin D receptor activator (VDRA) or a Vitamin D analog, preferably paricalcitol, to treat, prevent and delay progression of kidney disease.

### BACKGROUND OF THE INVENTION

The prevalence of end-stage renal disease (ESRD) is increasing at an alarming rate. In 2000, end stage kidney disease developed in over 90,000 people in the United States. The current population of patients on dialysis therapy or needing transplantation is 380,000 and projected to be 651,000 patients in 2010. Care for patients with ESRD already consumes more than $18 billion per year in the U.S, a substantial burden for the health care system. New data released in 2003 reported that 19.5 million U.S. adults have chronic kidney disease (CKD), and 13.6 million had Stage 2-5 CKD, as defined by the National Kidney Foundation Kidney Disease Outcomes Quality Initiative (NKF K/DOQI). Adverse outcomes of chronic kidney disease can often be prevented or delayed through early detection and treatment.

The pathogenesis for progression of renal fibrosis occurs through two mechanisms, which are additive: glomerulosclerosis and tubulointerstitial fibrosis (TIF). Insults to the glomerula from hemodynamic, immune or metabolic systems can injure endothelial, epithelial or mesangial cells in the kidney through the body's inflammatory and hemodynamic adaptive processes. As a result, mesangial cells proliferate, leading to glomerular fibrosis (glomerulosclerosis). This fibrotic mechanism causes proteinuria, increases cytokines and TGF-β, leading to nephron loss. Glomerulosclerosis decreases the glomerular filtration rate (GFR).

In humans, as GFR falls, kidney function and mass decline, even after the original disease becomes inactive. Surviving nephrons attempt to compensate by adapting their structure and function to meet excretory demands, leading to glomerular hyperfiltration and hypertrophy. Glomerular capillary hypertension is often maintained by angiotensin dependent mechanisms. Angiotensin II (AII) has emerged as a central mediator of the glomerular hemodynamic changes associated with progressive renal injury. This glomeruli hemodynamic adaptation further damages glomeruli and exacerbates glomerulosclerosis and nephron loss.

Angiotensin-converting enzyme inhibitors (ACEIs) and/or angiotensin receptor blockers (ARBs) plus/minus aldosterone blockade are the current regimen to treat hypertension (HTN), congestive heart failure (CHF), diabetic nephropathy (DN) and delay the progression of chronic kidney disease (CKD). Their effects on CKD are independent to their effects on controlling BP and treating HTN. In most cases, these therapies slowed the progression of CKD but did not arrest the decline to ESRD.

An important limitation of long-term use of ACEI and/or ARB is that these may lead to renin accumulation and the increase in downstream proteins, which may lead to an escape of ACE inhibition pathway with subsequent increase in AII and aldosterone generation. Aldosterone blockage in addition to ACEI and/or ARB to avoid aldosterone escape has additional benefit in the prevention of organ damage, but the renin level is still elevated in some patients. Additionally, incomplete arrest is explained by the fact that ACEI and ARB mainly target glomerular pathology and have weak effects on TIF.

TIF severity recently has been shown to correlate more highly with renal function than with glomerulosclerosis, resulting from a metabolic, immune or hemodynamic insult to the kidney. Renal TIF involves the following key and newly understood steps: 1) loss of adhesion of tubular epithelial cells and loss of cellular integrity by down regulation of E-cadherin; 2) transdifferentiation of tubular epithelial cells through de novo alpha-smooth muscle actin expression and actin reorganization of those epithelial cells that have lost adhesion; 3) disruption of the tubular basement membrane by increased matrix metalloproteinase (MMP) activity; and 4) transdifferentiated tubular cells that migrate and invade the interstitium, become myofibroblasts and cause fibrosis. Interruption of an early step in the pathway that leads to TIF could be an advantageous treatment. However, the market lacks such a medication.

It has been shown that the decreased serum vitamin D level correlates with decreased GFR and renal fibrosis. E. Ishimura, et al., Serum Levels of 1, 25 Dihydroxyvitamin D, 24,25 dihydroxyvitamin D, 25-hydroxyvitamin D in nondialyzed patients with chronic renal failure, Kidney International, Vol. 55 (1999) p. 1019-1027. However, the role of Vitamin D, if any, in the disease process itself has not been well understood before now. Researchers have studied whether VDRAs have a protective effect on the kidneys. Five recent studies have confirmed VDRAs can prevent glomerular injury and glomerulosclerosis. The studies claimed that vitamin D inhibits mesangium proliferation and inflammation, thereby ameliorating glomerular fibrosis.

Beyond effects on inflammation and proliferation, we believe that VDRAs can delay progression of chronic kidney disease by inhibiting renin secretion, which would prevent or reduce the ACE escape and the subsequent mesangial proliferation and glomerulosclerosis, and, more importantly, by preventing tubular interstitial fibrosis by blocking tubular epithelial to myofibroblast transdifferentiation.

Recent literature discloses that endogenous VDRA (calcitriol) could down regulate renin gene expression. See for example, Y. Li, et al., 1,25-Dihydroxyvitamin D₃ is a negative endocrine regulator of the renin-angiotensin system, J. Clin. Invest., July, 2002 (incorporated herein by reference). According to the present invention, down regulation of renin by VDRAs can prevent or can reduce ACE escape which will have an additive or synergistic effect to therapy with ACEI, ARB and/or aldosterone blockers in preventing glomerulosclerosis.

Besides targeting pathogenesis of glomerulosclerosis through RAAS, VDRAs could increase E-cadherin expression to keep the integrity of the tubular cells, could decrease alpha-smooth muscle actin expression to prevent epithelial to myofibroblast transdifferentiation and could decrease MMP activity to prevent tubular basement disruption and cell migration. The summary of these effects would result in blocking the tubular epithelial myofibroblast transdifferentiation and preventing TIF.

As shown in FIG. 1, the glomerular fibrosis pathway and the tubular interstitial fibrosis pathways are connected through effects on the renin-angiotensin (II)-aldosterone system (RAAS). We hypothesize that VDRAs prevent both glomerular and tubular interstitial fibrosis. In particular, VDRAs can be useful by their therapeutic action with respect to any of the following: 1) decreased inflammatory process; 2) decreased mesangial proliferation; 3) suppression of the renin-angiotensin-aldosterone system, especially renin production; 4) decreased glomerular hyperfiltration and hypertrophy; 5) decreased glomerular capillary pressure and single GFR; 6) decreased proteinuria; 7) reversal of abnormal cytokine activity; 8) decreased TGF-β activity; 9) increased E-cadherin; 10) decreased α-smooth muscle actin expression to prevent epithelial to myofibroblast transdifferentiation; 11) decreased matrix metalloproteinase activity; 12) inhibiting PAI-1 expression and 13) preventing increased renin, angiotensin II and aldosterone formation due to escape from the ACE inhibition and ARB therapy.

A multi-drug approach according to the present invention which blocks both pathways for renal disease progression would be advantageous. The present invention is therefore directed to advantageous combinations of a VDRA or Vitamin D analog with an ACE inhibitor and/or an angiotensin receptor blocker and/or aldosterone inhibitor.

### SUMMARY OF THE INVENTION

The present invention is directed to methods for preventing, treating and delaying progression of kidney disease, including chronic kidney disease and pharmaceutical compositions useful therefor. According to one embodiment, the present invention relates to VDRA/Vitamin D analog-containing compositions for preventing, treating and delaying progression of kidney disease.

According to other aspects of the present invention, the Vitamin D analog can be doxercalciferol or alfacalcidol.

Especially preferred compositions of the present invention include a VDRA/Vitamin D analog and one or more of the following agents: an angiotensin converting enzyme inhibitor (ACEI) or an angiotensin II receptor 1 (ARB) blocker or an aldosterone blocker.

According to other aspects of the invention, pharmaceutical compositions can be administered through a sustained (or continuous) delivery system. The present invention also contemplates other modes of administration, including but not limited to oral, injectable and transdermal.

### BRIEF DESCRIPTION OF THE DRAWINGS

FIG. 1 illustrates a Northern blot which evidences that paricalcitol treatment of As4.1-hVDR cells dose-dependently inhibits renin mRNA expression.

FIG. 2 illustrates the results of a renin promoter-luciferase assay used to examine the activity of paricalcitol to suppress renin gene transcription.

FIG. 3 illustrates the effect of paricalcitol and calcitriol on PAI-1 in primary culture of human coronary artery smooth muscle cells.

FIG. 4 illustrates urinary protein excretion in ApoE knockout mice

FIG. 5 illustrates changes in dipstick proteinuria from baseline to final visit.

FIG. 6 illustrates the decrease in dipstick proteinuria from baseline to final visit in ACE/ARB users

### DESCRIPTION OF THE EMBODIMENTS OF THE INVENTION

The present invention is generally directed to compositions containing a VDRA/Vitamin D analog to treat or prevent kidney disease, including chronic kidney disease. The present invention also relates to methods of treating kidney disease by administering to a patient a pharmaceutical composition containing a therapeutically effective amount of a VDRA/Vitamin D analog.

Treatment of patients with kidney disease by administration of a therapeutically effective amount of a VDRA/Vitamin D analog-containing composition according to the invention can be advantageous because the VDRA/Vitamin D analog can act at any one or all of the following points in the renal biochemical pathway:
decreased inflammation of cells;
decreased mesangial proliferation;
decreased activation of the renin-angiotensin-aldosterone system;
decreased hyperfiltration and hypertrophy;
decreased glomerular capillary pressure and single glomerular filtration rate;
decreased proteinuria;
reversal of abnormal cytokine profile;
decreased TGF-β levels;
increased E-cadherin, decreased α smooth muscle actin, decreased MMP;
decrease in PAI-1.

In contrast, conventional treatments based on administration of an ACEI (i.e., without a VDRA/Vitamin D analog), for example, only reduce angiotensin (II), but lack these other effects. Administration of ACEI may not be an attractive long term treatment due to adverse consequences.

According to some aspects of the present invention, the inventive compositions contain a VDRA/Vitamin D analog and at least one of the following agents: an ACE inhibitor, an angiotensin (II) receptor blocker (ARB) and aldosterone blocker in therapeutically effective amounts to inhibit renin production or inhibit activation of the renin-angiotensin-aldosterone system. Preferred compositions contain paricalcitol with at least one of these other agents. Such combinations can avoid ACE inhibition escape and aldosterone escape with subsequent increase in angiotensin (II) and aldosterone generation.

Suitable ACE inhibitors, ARB and aldosterone blockers are commercially available. Suitable ACE inhibitors include, but are not limited to: captopril (commercially available under the tradename CAPOTEN from Mylan), enalapril (commercially available under the tradename VASOTEC from Merck), fosinapril (commercially available under the tradename MONOPRIL from Bristol Myers Squibb), benzapril (commercially available under the tradename LOTENSIN from Novartis Pharmaceuticals), moexipril (commercially available under the tradename UNIVASC from Schwarz Pharma), perindopril (commercially available under the tradename ACEON from Solvay), quinapril (commercially available under the tradename ACCUPRIL from Parke-Davis), ramipril (commercially available under the tradename ALTACE from Monarch), trandolapril (commercially available under the tradename MAVIK from Abbott Laboratories of North Chicago, Ill.), lisinopril (commercially available under the tradenames PRINIVIL from and ZESTRIL from Astra Zeneca). Suitable angiotensin receptor blocking agents include, but are not limited to: losartan (commercially available as COZAAR from Merck), irbesartan (commercially available as AVAPRO from Bristol Myers Squibb and Sanofi), candesartan (commercially available as ATACAND from Astra Zeneca), eprosartan (commercially available as TEVETEN from Biovail Corporation of Canada), telmisartan (commercially available as MICARDIS from Boehringer Ingelheim) and valsartan (commercially available as DIOVAN from Novartis).

Suitable aldosterone blockers include, but are not limited to: eplerenone (commercially available under the tradename INSPRA from Pharmacia), spironolactone (commercially available under the tradenames Aldactone, Adultmin, Aldopur, Aldospirone, Almatol, Berlactone, Diatensec, Diram, Esekon, Hypazon, Idrolattone, Merabis, Novospiroton, Osiren, Osyrol, Pirolacton, Resacton, Sincomen, Spiractin, Spiroctan, Spirolacton, Spirolang, Spironex, Spirotone, Tevaspirone, Verospiron, Xenalon Lactabs, Youlactone).

Additional components, e.g., physiologically acceptable carriers, solvents, binders, antioxidants, colorants, substrates can be used as necessary or desired.

Preferred treatment or preventive regimens for patients with kidney disease according to the present invention would administer therapeutically effective VDRA/vitamin D analog-containing compositions according to the invention for a sufficient period to effect sustained or continuous delivery. As used herein, a “therapeutically effective dose” is a dose which in susceptible subjects is sufficient to prevent progression or cause regression of kidney disease or which is capable of relieving the symptoms caused by kidney disease.

An exemplary dosing regimen would provide the equivalent of 0.5 micrograms of calcitriol per day or at least about 1 microgram calcitriol by three times weekly. For paricalcitol, a suitable dosing regimen would provide the equivalent of about 4 micrograms paricalcitol daily or at least about 4 micrograms paricalcitol three times weekly. Suitable dosing regimens for other VDRA/Vitamin D analogs, e.g., doxercalciferol, can be determined straightforwardly by those skilled in the art based on the therapeutic efficacy of the VDRA/Vitamin D analog to be administered.

Since ACEI, ARB and aldosterone inhibitors have different efficacies and affect the body through different proteins in the rennin-angiotensin-aldosterone system (RAAS) pathway than a VDRA/Vitamin D does, compositions according to the present invention can incorporate an ACEI, ARB or aldosterone inhibitor to be administered according to conventional dosing regimens, which are well known and readily available to those skilled in the art.

The invention also contemplates continuous or sustained drug delivery forms containing the selected VDRA/Vitamin D analog, and an ACEI and/or an ARB and/or an aldosterone blocker. Suitable delivery forms include, but are not limited to, tablets or capsules for oral administration, injections, transdermal patches for topical administration (e.g., drug to be delivered is mixed with polymer matrix adhered to or absorbed on a support or backing substrate, e.g., ethylcellulose), depots (e.g., injectable microspheres containing the desired bioactive compounds) and implants. Techniques for making these drug delivery forms are well-known to those skilled in the art.

### EXAMPLES

### Example 1

### Activity of Paricalcitol to Suppress Renin Expression

Recently, it has been found that 1,25-dihydroxyvitamin D functions as a negative regulator of renin biosynthesis in vitro and in in vivo studies. Calcitriol is able to inhibit renin gene expression, which provides a molecular basis to explore the use of vitamin D and vitamin D analogs as new renin inhibitor to regulate rennin-angiotensin-aldosterone system (RAAS).

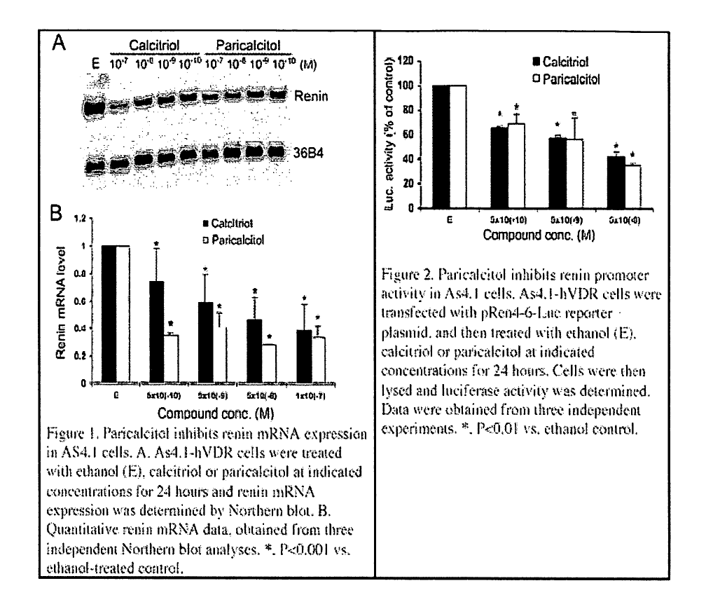

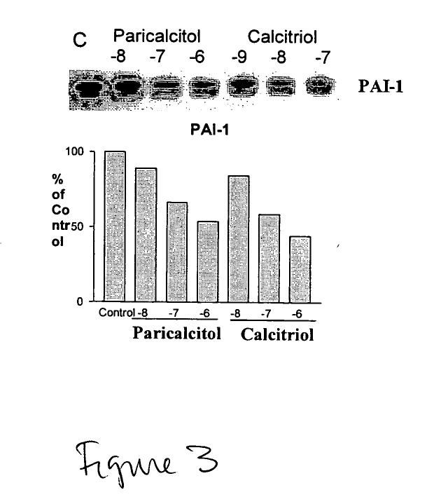

Using an in vitro cell culture system, the activity of paricalcitol to suppress renin gene expression was examined using previously published techniques (1,25-Dihydroxyvitamin D₃ is a negative endocrine regulator of the renin-angiotensin system, J. Clin. Invest., July 2002). As shown in FIG. 1, by Northern blot analysis, paricalcitol treatment of As4.1-hVDR cells dose-dependently inhibits renin mRNA expression. In fact, its renin-inhibiting activity appears a bit more potent than calcitriol (FIGS. 1A and B). This inhibitory effect is confirmed by renin promoter-luciferase reporter assays, which examine the activity of paricalcitol to suppress renin gene transcription. In these assays, paricalcitol appears at least as potent as calcitriol to suppressing the activity of the renin gene promoter (FIG. 2).

### Example 2

### Effect of VDR Activators on PAI-1

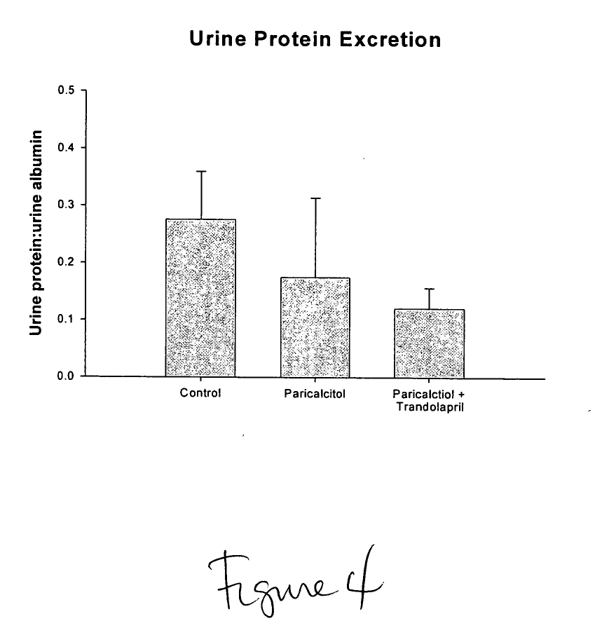

The effect of paricalcitol and calcitriol on PAI-1 in primary culture of human coronary artery smooth muscle cells was investigated. (See FIG. 4.) PAI-1 (plasminogen activator inhibitor type-1) is one of the risk markers for coronary heart disease, and is enhanced in atherosclerotic plague and colocalized with macrophages. Human coronary artery smooth muscle cells were incubated with paricalcitol or calcitriol at the indicated concentration for 24 hr at 37° C. Samples were solubilized in SDS-PAGE sample buffer, and the protein content in each sample was determined by the Bio-Rad dye-binding protein assay. Samples were resolved by SDS-PAGE using a 4-12% gel, and proteins were electrophoretically transferred to PVDF membrane for Western blotting.

The membrane was blotted for 1 h at 25° C. with 5% nonfat dry milk in PBS-T and then incubated with a mouse anti-PAI-1 monoclonal antibody in PBS-T overnight at 4° C. The membrane was washed with PBS-T and incubated with a horseradish peroxidase-labeled anti-rabbit antibody for 1 h at 25° C. The membrane was then incubated with detection reagent (SuperSignal WestPico). The specific bands were visualized by exposing the paper to Kodak BioMax films.

FIG. 4 shows the results from Western blot using an anti-PAI-1 antibody. Two observations may be noted in these studies: (1) 100% inhibition of growth was never achieved even at 1 μM of any of the test compound. Confocal microscopy studies confirm that, although these drugs are potent in inducing the translocation of VDR from cytoplasm to nucleus, not all cells respond to VDRAs even after 2 h of exposure, which may explain the <100% inhibition. (2) Although paricalcitol is known to be less potent than calcitriol in the clinical studies, it exhibits similar potency to calcitriol in this assay. By checking the effect of drugs on the expression of 24(OH)ase, it was found that paricalcitol is less potent than calcitriol on stimulating the expression of 24(OH)ase, which may partially explain the higher potency of paricalcitol in this assay.

These results show that paricalcitol and calcitriol are equally potent in reducing the PAI level in human coronary artery smooth muscle cells. Paricalcitol is usually dosed approximately 4 fold higher than calcitriol in the clinical situation, which may translate into a 4-fold higher potency in regulating the function of smooth muscle cells.

In fibrotic renal disease, PAI-1 is increased and localizes to areas of glomerulosclerosis. Conversely, inhibition of angiotensin or aldosterone decreases PAI-1 and also decreases renal scarring. These results show that paricalcitol is able to decrease PAI-1 level, suggesting the potential role of paricalcitol on attenuation of glomerulosclerosis.

### Example 3

### Effect of VDR Activator Alone and in Combination with ACEI, on Nephropathy in Rodent Model of CKD

Using the ApoE KO mouse after uninephrectomy, which is an accepted model of CVD and CKD, the effect of paricalcitol and trandolapril to treat and to delay progression of kidney disease was examined. Animals received daily subcutaneous paricalcitol (30 mg/100 g body weight) or paricalcitol (30 mg/100 g body weight)+trandolapril (1 mg/kg) for 12 weeks. Proteinuria or albuminuria are established risk factors for progressive loss of kidney function. Since urinary protein excretion correlates with kidney damage, at the end of the 12 week treatment period mice were placed in metabolic cages for measurement of 48-hr urine albumin and creatinine excretion by ELISA assays. Urinary albumin excretion is expressed as mg albumin/mg creatinine.

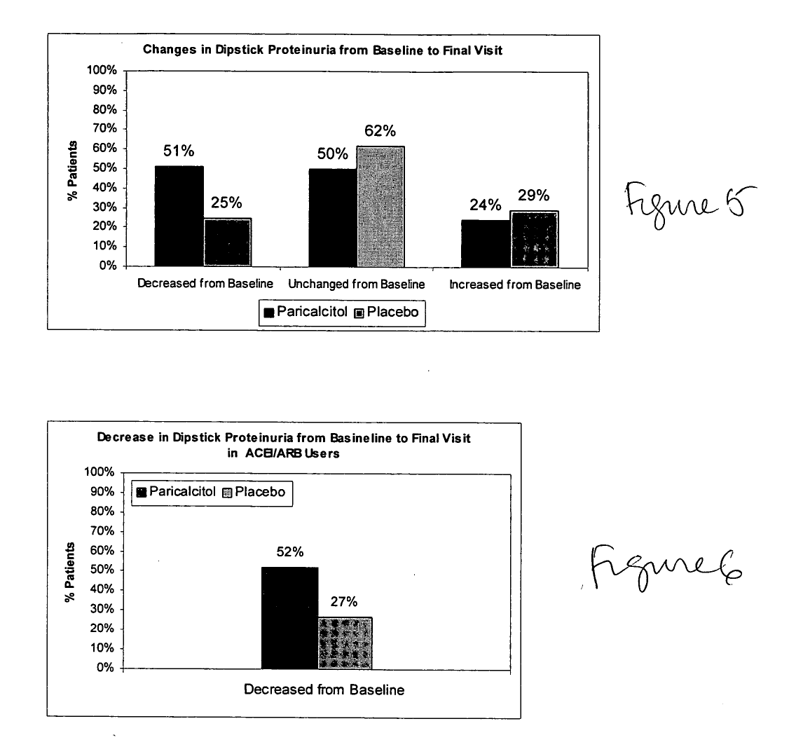

FIG. 5 provides the results for urinary albumin excretion, represented as a ratio to urinary creatinine excretion. These results show that paricalcitol lowers urinary albumin excretion and that the combination of paricalcitol and trandolapril further decreases urinary albumin excretion. The observed reduction in albuminuria associated with paricalcitol treatment and the synergistic effects of RAAS inhibition on urinary albumin excretion supports the potential renal protective benefit of paricalcitol and of paricalcitol in combination with RAAS inhibitors.

### Example 4

Paricalcitol capsule, a synthetic 3^{rd} generation vitamin D analog, and selective vitamin receptor activator (SVDRA) was evaluated in three randomized, double-blind, placebo-controlled, multicenter studies in CKD Stage 3 and 4 patients with SHPT. Two studies dosed three times a week(TIW), no more often than every other day, and 1 study was conducted with once a day dosing regimen (QD). Data from these three studies were combined and analyzed for the following safety parameters:
Calcium and phosphorus metabolism, kidney function parameters and adverse event profile
Changes in dipstick proteinuria
   Method:
Randomized, double-blind, placebo controlled, multicenter studies with a Pre-treatment/Washout Phase of 1-4 weeks and a Treatment Phase of 24 weeks
Patients were randomized 1:1 to paricalcitol or placebo
Major entry criteria:
   Age≧18 years
   CKD patients with eGFR (MDRD formula) 15-60 mL/min/1.73 m²
   Average of the last 2 iPTH values≧150 pg/mL, all values≧120 pg/mL (iPTH measured by Nichols first generation intact PTH assay)
   2 consecutive serum calcium values between 8.0-10.0 mg/dL, and 2 consecutive serum phosphorus values≦5.2 mg/dL
   Spot urinary calcium-to-creatinine ratio<0.2
   Not taking maintenance calcitonin, bisphosphonates or drugs that may affect calcium or bone metabolism.
   Not taking glucocorticoids for >14 days within the last 6 months.
      Concurrent Phosphate Binder Usage

Same brand and stable dose of phosphate binders for 4 weeks prior to enrollment.

Patients were to remain on the same brand and stable dose of phosphate binders throughout the study

Study Drug Administration:

Initial Dose: 2 or 4 mcg for TIW regimen and 1 or 2 mcg for QD regimen

Dose Increment: 2 mcg for TIW regimen and 1 mcg for QD regimen

Dose increases every 4 weeks, dose decreases every 2 weeks or sooner

Results

Patient Demographics

Overall, 220 patients across the 3 studies (107 paricalcitol capsule and 113 placebo) enrolled in the study in a 1:1 randomization. Patient demographics are presented in Table 1.

TABLE 1
Patient Demographics and Disposition (All Treated Patients)

| | Paricalcitol | |
|---|---|---|
| | Capsule | Placebo |
| | (N = 107) | (N = 113) |
| Gender | | |
| Female | 32% | 33% |
| Male | 68% | 67% |
| Race | | |
| White | 69% | 73% |
| Black | 26% | 26% |
| Other | 5% | 1% |
| Age (years) | | |
| Mean (SE) | 64 (1) | 62 (1) |
| Range | 22-91 | 32-93 |
| Baseline eGFR | | |
| (mL/min/1.73 m²) | | |
| Mean (SE) | 23.1 (0.78) | 23.0 (0.73) |
| Range | 10.0-55.1 | 13.0-49.0 |
| Time Since CKD | | |
| Diagnosis (years) | | |
| Mean (SE) | 5.4 (0.63) | 6.1 (0.71) |
| Range | 0.2-51.4 | 0.2-38.7 |
| Completed 24 weeks | 77% | 83% |
| of Treatment | | |

TABLE 2
Paricalcitol Capsule Exposure

| | Paricalcitol (N = 107) |
|---|---|
| Overall Average Weekly Dose (mcg/wk) | 9.5 (3.81) |
| Maximum Weekly Dose (mcg/wk) | 36 |
| Overall Average Daily Dose (mcg/day) | 1.4 |
| Proportion of patients treated for 113 days | 83% |
| or longer | |
| Overall paricalcitol exposure | 43.2 patient-years |

Phosphate Binder Usage

The majority of patients were not receiving any phosphate binders during the study. At baseline, only 21% and 24% of paricalcitol and placebo patients, respectively, were receiving calcium-based phosphate binders and/or calcium supplements, and two patients in each treatment group were taking sevelamer. Eighty-three percent of paricalcitol patients and 90% of placebo patients maintained the same type and dosage of phosphate binders throughout the study. Of patients who did not receive phosphate binders and/or calcium supplement at baseline, 16% in the paricalcitol group and 11% in the placebo group initiated such medications during the treatment period.

TABLE 3
Phosphate Binder Usage

| | |
|---|---|
| Paricalcitol | Placebo |
| (N = 107) | (N = 113) |

At Baseline

| | | |
|---|---|---|
| Calcium based phosphate | 22 (21%) | 27 (24%) |
| binder | | |
| Sevelamer hydrochloride | 2 (2%) | 2 (2%) |
| Non-User | 83 (78%) | 84 (74%) |

During the Treatment

| | | |
|---|---|---|
| Dose unchanged | 83% | 90% |
| Dose Decreased | 12.5% | 3% |
| Dose Increased | 4.5% | 7% |
| Initiated phosphate binder | 17% | 12% |

Changes in Dipstick Proteinuria

Changes in proteinuria, measured by automated semiquantitative dipstick testing, were evaluated in 195 CKD Stage 3 and 4 patients. Among them, 43.6% (41/94) of paricalcitol patients and 48.5% (49/101) of placebo patients were diabetic. Patient disposition in dipstick proteinuria analysis is presented in table 4.

TABLE 4
Patient Disposition in Dipstick Proteinuria Analysis

| | Paricalcitol | Placebo |
|---|---|---|
| Number of patients | 94 | 101 |
| who had dipstick proteinuria data | | |
| Number of patients who | 57 | 61 |
| had proteinuria at baseline | | |
| Number of patients who | 76 | 77 |
| could have an increase in proteinuria | | |
| Number of patients who were | 42 | 41 |
| on ACEi/ARB therapy and | | |
| had proteinuria at baseline | | |

Differences between treatment groups with respect to proteinuria were examined in change from baseline to final visit analyses. Among patients who had proteinuria at baseline, 51% (29/57) of paricalcitol-treated patients experienced a reduction in proteinuria compared to 25% (15/61) placebo patients (p=0.004). Of patients who could have an increase in proteinuria from baseline, 24% (18/76) paricalcitol-treated patients experienced an increase compared to 29% (23/77) for placebo patients (p=0.466) (FIG. 5)

FIG. 6 examines the additive effect of paricalcitol to the effect on inhibiting the RAAS system in those patients receiving ACEIs or ARBs. Analysis revealed that of patients who were on ACEI/ARB therapy and had proteinuria at baseline, 52% (22/42) paricalcitol patients vs. 27% (11/41) placebo patients had a decrease in proteinuria (p=0.025).

These data suggests a beneficial effect of paricalcitol on reduction of dipstick proteinuria, which is independent of PTH control, not limited to diabetics and synergistic to ACEi/ARB therapy.

The reduction in dipstick proteinuria associated with paricalcitol treatment suggests a potential beneficial effect of paricalcitol on renal protection and on the delay of CKD progression. This observed benefit supports the potential renal protective role of paricalcitol, which in this study, was not limited to diabetics and was synergistic to ACEI/ARB therapy.

1. A sustained release pharmaceutical composition for preventing, treating and delaying progression of kidney disease, including chronic kidney disease, comprising:
a therapeutically effective amount of Vitamin D receptor activator or Vitamin D analog; and optionally
a therapeutically effective amount of at least one member of the group consisting of an angiotensin converting enzyme inhibitor, an angiotensin (II) receptor (I) blocker, and an aldosterone blocker.

2. A sustained release pharmaceutical composition according to claim 1, wherein said Vitamin D analog is selected from the group consisting of calcitriol and doxercalciferol.

4. A sustained release pharmaceutical composition according to claim 1, wherein said Vitamin D receptor activator is paricalcitol.

5. A sustained release pharmaceutical composition according to claim 1 is a transdermal patch.

6. A sustained release pharmaceutical composition according to claim 1 is an oral dosage form.

7. A sustained release pharmaceutical composition according to claim 1 is a subcutaneous dosage form.

8. A sustained release pharmaceutical composition according to claim 1 is an injectable dosage form.

9. A sustained release pharmaceutical composition according to claim 8, wherein said injectable dosage form is a member of the group consisting of a subcutaneous dosage form and a depot dosage form.

10. A sustained release pharmaceutical composition according to claim 7 is an implantable form.

11. A pharmaceutical composition for treating, preventing or delaying progression of kidney disease, including chronic kidney disease, in a mammal, comprising:
a therapeutically effective amount of a Vitamin D receptor activator or a Vitamin D analog; and
an optional therapeutically effective amount of at least one member of the group consisting of an angiotensin converting enzyme inhibitor, an angiotensin (II) receptor (I) blocker, and an aldosterone blocker

12. A pharmaceutical composition according to claim 11, wherein said Vitamin D analog is selected from the group consisting of paricalcitol, calcitriol, and doxercalciferol.

13. A pharmaceutical composition according to claim 11 is a transdermal patch.

14. A pharmaceutical composition according to claim 11 is an oral dosage form.

15. A pharmaceutical composition according to claim 11 is a subcutaneous dosage form.

16. A pharmaceutical composition according to claim 11 is an injectable dosage form.

17. A pharmaceutical composition according to claim 16, wherein said injectable dosage form is a member of the group consisting of a subcutaneous dosage form and a depot dosage form.

18. A pharmaceutical composition according to claim 15 is an implantable form.

19. A method of preventing, treating and delaying progression of kidney disease, including chronic kidney disease, in a mammal, comprising the step of administering to said mammal a pharmaceutical composition according to claim 9.

20. A method according to claim 19, wherein the administering step is continuous.

21. A method according to claim 19, wherein the administering step is carried out using a transdermal patch.

22. A method according to claim 19, wherein the administering step is carried out using an oral dosage form.

23. A method according to claim 19, wherein the administering step is carried out using an injectable dosage form.

24. A method according to claim 19, wherein the administering step is carried out using a subcutaneous dosage form.
